(19) United States
(12) Patent Application Publication
(10) Pub. No.: US 2006/0074491 A1
(43) Pub. Date: Apr. 6, 2006

(54) Boronized medical implants and process for producing the same

(75) Inventors: Bryan Smith, Fort Wayne, IN (US); Sarah Aust, Warsaw, IN (US); Ron Overholser, Warsaw, IN (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: DePuy Products, Inc., Warsaw, IN (US)

(21) Appl. No.: 10/954,677

(22) Filed: Sep. 30, 2004

(51) Int. Cl. A61F 2/32; A61F 2/38; A61F 2/28

(52) U.S. Cl. 623/20.28; 623/22.15; 623/23.53; 148/239

(57) ABSTRACT

The invention provides metallic medical implants or medical implant parts having a bearing surface comprising a boronized metal layer. The invention further provides a process for producing a medical implant or medical implant part comprising a boronized metal layer.

### FIELD OF THE INVENTION

This invention pertains to metallic medical implants or medical implant parts comprising a boronized metal layer and processes for producing such medical implants or medical implant parts.

### BACKGROUND OF THE INVENTION

Orthopaedic implants typically must endure significant mechanical stresses and an in vivo environment intent on attacking most foreign materials introduced into a patient's body. Therefore, the materials from which such orthopaedic implants are made must combine high strength, corrosion resistance, and tissue compatibility. Furthermore, due to the rigors often associated with revision surgery, it is desirable for the in vivo lifespan of an orthopaedic implant to be equal to or exceed the expected remaining lifespan of the recipient of the implant.

One of the variables affecting the longevity of load-bearing orthopaedic implants, such as hip-joint implants, is the rate of wear of the implant's articulating surfaces. A typical hip-joint implant includes a femoral stem, a femoral head attached to the stem, and an acetabular cup against which the femoral head articulates. Wearing of these articulating surfaces generates debris particles that are released into the tissues surrounding the implant. It is generally accepted by orthopaedic surgeons and biomaterials scientists that these debris particles contribute, at least in part, to bone loss at the interface of the orthopaedic implant and the host bone. Indeed, the reaction of the body to these particles includes inflammation and deterioration of the tissues, particularly the bone to which the orthopaedic implant is anchored, through a process known as osteolysis. As the osteolysis progresses, the orthopaedic implant may become painfully loose and require revision.

The rate of wear of the articulating surfaces of orthopaedic implants is dependent upon a number of factors. These factors include, but are not limited to, the relative hardness and surface finish of the materials from which the articulating surfaces are made, the coefficient of friction between the materials of the articulating surfaces, the load applied to the articulating surfaces, and the stresses generated at the articulating surfaces. In an effort to decrease the rate of wear of the articulating surfaces of orthopaedic implants, and thereby extend the in vivo lifespan of such implants, several attempts have been made to address one or more of the above-identified factors which affect the rate of wear of such articulating surfaces. For example, orthopaedic implants have been developed which are made from relatively hard, wear-resistant, chemically inert oxide ceramics. However, ceramic implants often are brittle and lack the toughness of metallic implants, which can increase the risk of fracture. The brittleness and low toughness of ceramic implants also limits the use of such implants in certain applications, such as the femoral component of a knee arthroplasty. Furthermore, ceramic implants are not compatible with the beaded, porous ingrowth structures used to aid biologic fixation of implants implanted into patients without the use of bone cement.

U.S. Pat. No. 5,037,438 describes a prosthetic implant having a coating of blue-black or black zirconium oxide on the bearing surface of the prosthesis body. While the aforementioned patent claims that the coating produces a low friction, wear-resistant bearing surface, attempts to provide surface layers of zirconium oxide greater than approximately 8-10 microns in thickness have resulted in delamination of the zirconium oxide layer from the zirconium alloy substrate. The relatively thin coatings produced by the process may have limited abrasion or scratch resistance and may not be suitable for high contact stress applications, such as metal on metal hip bearings.

Other efforts aimed at increasing the wear performance of orthopaedic implants have included ion bombardment of the implant's articulating surfaces (e.g., nitrogen ion implantation), nitriding the implant's articulating surfaces, coating the articulating surfaces with diamond-like carbon or titanium nitride coatings, and oxygen diffusion hardening of, for example, titanium alloy implants. While each of these techniques is capable of producing a hardened articulating surface on the orthopaedic implant, some of the surface coatings or layers produced by such techniques suffer from limited adhesion to the substrate. Furthermore, some of the techniques are only capable of producing very thin coatings or layers of the hardened material, and others produce coatings or layers exhibiting peak hardness values that are not relatively high.

A need therefore exists for metallic orthopaedic implants or implant parts having hardened, wear-resistant articulating surfaces. A need also exists for a process for producing orthopaedic implants or implant parts comprising such hardened, wear resistant articulating surfaces. The invention provides such orthopaedic implants and implant parts, as well as a process for producing the same. These and other advantages of the invention, as well as additional inventive features, will be apparent from the description of the invention provided herein.

### BRIEF SUMMARY OF THE INVENTION

The invention provides a medical implant or medical implant part comprising (a) a metallic body comprising a metal or metal alloy, and (b) a bearing surface disposed on the body, the bearing surface comprising a boronized layer of the metal or metal alloy.

The invention also provides a medical implant for implantation into a patient, the medical implant comprising (a) a femoral component for replacing one or more of the patient's femoral condyles, the femoral component having a metallic body comprising a metal or metal alloy and a bearing surface disposed on the body, the bearing surface comprising a boronized layer of the metal or metal alloy, (b) a tibial component for replacing at least a portion of the patient's proximal tibial articular surface, and (c) a polymeric bearing component which rests on the tibial component and confronts the bearing surface of the femoral component.

The invention also provides a medical implant for implantation into a patient, the medical implant comprising (a) a femoral stem for anchoring the implant into the patient's femur, (b) a femoral head which attaches to the upper end of the femoral stem, the femoral head having a metallic body comprising a metal or metal alloy and a bearing surface disposed on the body, the bearing surface comprising a boronized layer of the metal or metal alloy, and (c) an acetabular component for replacing the patient's acetabulum, the acetabular component comprising a liner which confronts the bearing surface of the femoral head.

The invention further provides a process for producing a medical implant or medical implant part, the process comprising the steps of (a) providing a medical implant or medical implant part having a metallic body, (b) providing a boronizing agent which yields boron upon heating, (c) heating the boronizing agent to a temperature at which the boronizing agent yields boron, (d) contacting at least a portion of the metallic body with the boron produced by the boronizing agent, and (e) heating the medical implant or medical implant part to an elevated temperature for a time sufficient for at least a portion of the boron produced by the boronizing agent to diffuse into at least a portion of the metallic body of the medical implant or medical implant part.

### DETAILED DESCRIPTION OF THE INVENTION

The invention provides a medical implant or medical implant part comprising (a) a metallic body comprising a metal or metal alloy and (b) a bearing surface disposed on the body.

The medical implant or medical implant part can be any suitable metallic medical implant or medical implant part. Suitable implants or implant parts include, but are not limited to, the femoral component (e.g., the component which replaces one or more of the patient's femoral condyles) of an uni-compartmental knee arthroplasty or a total knee arthroplasty, the tibial component (e.g., the component which replaces at least a portion of the patient's proximal tibial articular surface or tibial plateau) of an uni-compartmental knee arthroplasty or a total knee arthroplasty, the femoral head of a hip arthroplasty, the acetabular cup or liner of a hip arthroplasty, the humeral head of a shoulder arthroplasty, the humeral or ulnar component of an elbow arthroplasty, the metacarpal or radial stem of a wrist arthroplasty, the vertebral endplate components of a disc arthroplasty (e.g., a cervical vertebral disc arthroplasty), and the tibial or talar component of an ankle arthroplasty.

The medical implant or medical implant part of the invention comprises a metallic body. The metallic body of the implant or implant part can comprise, consist essentially of, or consist of any suitable metal or metal alloy (e.g., any metal which readily forms hard borides upon diffusion of boron into the surface at elevated temperatures). For example, the metallic body can comprise a metal or metal alloy selected from the group consisting of cobalt, cobalt alloys, titanium, titanium alloys, and mixtures thereof. Preferably, the metal or metal alloy is selected from the group consisting of cobalt, cobalt-chromium alloys, titanium, titanium-aluminum alloys, and mixtures thereof. Suitable cobalt-chromium alloys include, but are not limited to, the cast, forged, and wrought cobalt-28-chromium-6-molydenum (Co28Cr6Mo) alloys described in, for example, ASTM Standards F75-01, F799-02, and F1537-00, respectively. Suitable titanium-aluminum alloys include, but are not limited to, the titanium-3-aluminum-2.5-vanadium alloy (Ti3Al2.5V) described in, for example, ASTM Standard F2146-01 and the titanium-6-aluminum-4-vanadium (Ti6Al4V) alloy described in, for example, ASTM Standard F136-02a.

The medical implant or medical implant part comprises a bearing surface disposed on the body. As utilized herein, the term “bearing surface” is used to refer to a portion of the surface of a medical implant or medical implant part which articulately or movably confronts another surface (e.g., the surface of another medical implant or medical implant part) when the medical implant or medical implant part is implanted in a patient. For example, the bearing surface of the medical implant or medical implant part can correspond to the outer surface of a femoral component of a uni-compartmental or total knee arthroplasty, which surface confronts the polymeric bearing component of the arthroplasty. Alternatively, the bearing surface of the medical implant or medical implant part can correspond to the outer surface of the femoral head of a hip arthroplasty, which surface confronts the liner of the acetabular cup.

The bearing surface of the medical implant or medical implant part preferably comprises a boronized layer of the metal or metal alloy from which the body of the implant or implant part is comprised. As utilized herein, the term “boronized” refers to a portion of the metal or metal alloy which comprises boron atoms that have diffused into the metal or metal alloy. For example, the boronized layer can comprise a mixture of borides. Such borides can have, for example, the formula MeB, MeB₂, or Me₂B, wherein Me represents a metal present in the body of the medical implant or medical implant part. For example, when the medical implant or medical implant part comprises Co28Cr6Mo alloy, the boronized layer can comprise a mixture of borides having the formula CoB, Co₂B, as well as other borides of cobalt, chromium, and/or molybdenum. When the medical implant or medical implant part comprises a titanium-aluminum-vanadium alloy (e.g., Ti3Al2.5V or Ti6Al4V), the boronized layer can comprise a mixture of borides having the formula TiB, TiB₂, as well as other borides of titanium, aluminum, and/or vanadium. Alternatively, the boronized layer can comprise boron atoms that have diffused into the lattice structure of the metal or metal alloy. In such boronized layers, the relatively small boron atoms typically fill a portion of the interstitial spaces (i.e., the spaces between adjacent metal atoms) present in the lattice structure of the metal or metal alloy. As will be understood by those of ordinary skill in the art, the composition of the boronized layer can be different at various points in the boronized layer (e.g., at various depths in the boronized layer). For example, the boronized layer of a medical implant or medical implant part according to the invention can predominantly comprise borides having the formula MeB or MeB₂ in the portion of the boronized layer closest to the surface of the metallic body, while predominantly comprising borides having the formula Me₂B or boron atoms filling a portion of the interstitial spaces in the lattice structure of the metal or metal alloy in the portion of the boronized layer furthest from the surface of the metallic body.

The boronized layer of the metal or metal alloy can have any suitable thickness. Typically, the boronized layer has a thickness of about 1 μm or more (i.e., the boronized layer extends at least about 1 μm below the surface of the metallic body). Preferably, the boronized layer has a thickness of about 2 μm or more, more preferably about 3 μm or more, and most preferably about 5 μm or more (e.g., about 6 μm or more, or about 8 μm or more). The boronized layer typically has a thickness of about 75 μm or less (e.g., about 70 μm or less, about 65 μm or less, about 60 μm or less, about 50 μm or less, or about 40 μm or less). In certain embodiments, the boronized layer preferably has a thickness of about 5 μm to about 30 μm (e.g., about 6 μm to about 30 μm, about 8 μm to about 30 μm, or about 15 μm to about 25 μm).

The boronized layer of the metal or metal alloy typically is harder than the untreated metal or metal alloy (e.g., the metal or metal alloy prior to boronizing). Preferably, the boronized layer of the metal or metal alloy has a higher Knoop hardness than the untreated metal or metal alloy. The Knoop hardness of the boronized layer can be determined using any suitable technique. Typically, the Knoop hardness of the boronized layer is determined using the technique described in ASTM Standard E384-99e1. In certain embodiments, the boronized layer of the metal or metal alloy has a Knoop hardness of about 1000 HK₅₀ or more, preferably about 1500 HK₅₀ or more, more preferably about 1750 HK₅₀ or more, and most preferably about 2000 HK₅₀ or more (e.g., about 2100 HK₅₀ or more).

In a preferred embodiment, the invention provides a knee arthroplasty implant (e.g., a uni-compartmental knee arthroplasty implant or a total knee arthroplasty implant). In such an embodiment, the medical implant preferably comprises (a) a femoral component for replacing one or more of the patient's femoral condyles, the femoral component having a metallic body comprising a metal or metal alloy and a bearing surface disposed on the body, the bearing surface comprising a boronized layer of the metal or metal alloy, (b) a tibial component for replacing at least a portion of the patient's proximal tibial articular surface, and (c) a polymeric bearing component which rests on the tibial component and confronts the bearing surface of the femoral component. The characteristics of this embodiment of the medical implant or medical implant part of the invention (e.g., the composition of the metallic body, the composition of the boronized layer, the thickness of the boronized layer, the hardness of the boronized layer, etc.) can be the same as those set forth above.

In another preferred embodiment, the invention provides a hip arthroplasty implant. In such an embodiment, the medical implant preferably comprises (a) a femoral stem for anchoring the implant into the patient's femur, (b) a femoral head which attaches to the upper end of the femoral stem, the femoral head having a metallic body comprising a metal or metal alloy and a bearing surface disposed on the body, the bearing surface comprising a boronized layer of the metal or metal alloy, and (c) an acetabular component for replacing the patient's acetabulum, the acetabular component comprising a liner which confronts the bearing surface of the femoral head. The characteristics of this embodiment of the medical implant or medical implant part of the invention (e.g., the composition of the metallic body, the composition of the boronized layer, the thickness of the boronized layer, the hardness of the boronized layer, etc.) can be the same as those set forth above.

The inventive medical implant or medical implant part can be prepared in any suitable manner. For example, the inventive medical implant or medical implant part can be prepared using methods similar to those used to boronize other types of metals, such as the process described in Knotek et al., “Surface Layers on Cobalt Base Alloys by Boron Diffusion,” Thin Solid Films, 45:331-339 (1977).

The invention further provides a preferred process for producing a medical implant or medical implant part comprising a boronized layer of a metal or metal alloy. In particular, the process comprises the steps of (a) providing a medical implant or medical implant part having a metallic body, (b) providing a boronizing agent which yields boron upon heating, (c) heating the boronizing agent to a temperature at which the boronizing agent yields boron, (d) contacting at least a portion of the metallic body with the boron produced by the boronizing agent, and (e) heating the medical implant or medical implant part to an elevated temperature for a time sufficient for at least a portion of the boron produced by the boronizing agent to diffuse into at least a portion of the metallic body of the medical implant or medical implant part.

The medical implant or medical implant part to be subjected to the process of the invention can be any suitable metallic medical implant or medical implant part. Suitable implants or implant parts include, but are not limited to, the femoral component (e.g., the component which replaces one or more of the patient's femoral condyles) of an uni-compartmental knee arthroplasty or a total knee arthroplasty, the tibial component (e.g., the component which replaces at least a portion of the patient's proximal tibial articular surface or tibial plateau) of an uni-compartmental knee arthroplasty or a total knee arthroplasty, the femoral head of a hip arthroplasty, the acetabular cup or liner of a hip arthroplasty, the humeral head of a shoulder arthroplasty, the humeral or ulnar component of an elbow arthroplasty, the metacarpal or radial stem of a wrist arthroplasty, the vertebral endplate components of a disc arthroplasty (e.g., a cervical vertebral disc arthroplasty), and the tibial or talar component of an ankle arthroplasty.

The process of the invention utilizes a boronizing agent which yields boron when it is heated. Certain boronizing agents can produce elemental boron (e.g., gaseous elemental boron) upon heating. Other boronizing agents can produce, upon heating, boron compounds that are capable of releasing one or more boron atoms into the metal or metal alloy when they contact the metallic body at elevated temperatures. Accordingly, the boronizing agent comprises a source of boron. The source of boron can be any suitable source which is capable of yielding boron (e.g., boron which is available to diffuse into the metallic body of the medical implant or medical implant part) upon heating. Suitable sources of boron include, but are not limited to, amorphous boron, crystalline boron, boron trifluoride (BF₃), boron trichloride (BCl₃), boron tribromide (BBr₃), diborane (B₂H₆), trimethyl boride ((CH₃)₃B), triethyl boride ((C₂H₅)₃B), boron carbide (B₄C), borax (Na₂B₄O₇.10H₂O or Na₂B₄O₇), metaboric acid (HBO₂), sodium borofluoride (NaBF₄), boric acid anhydride (B₂O₃), ferroboron, metal borides, and combinations thereof.

The boronizing agent also can comprise other substances that improve the agent's ability to produce boron for the boronizing process or improve the handling characteristics of the boronizing agent. For example, the boronizing agent can comprise any suitable filler, such as, carbon black, silicon carbide, aluminum oxides, magnesium oxides, silicon oxides, silicates, non-boridable metals, and combinations thereof. The boronizing agent also can comprise any suitable activator, such as, a fluoroboride (e.g., a tetrafluoroborate). The process of the invention can utilize a commercially available boronizing agent, which typically comprises a combination of a boron source, a filler, and an activator. Suitable commercially available boronizing agents include, but are not limited to, the EKABOR™ boronizing agents sold by BorTec GmbH.

Any suitable amount of the boronizing agent can be used in the inventive process. For example, when the boronizing agent is provided in the form of a powder, the medical implant or medical implant part can be packed into an excess of the boronizing agent such that the boronizing agent contacts only those portions of the metallic body that are to be boronized. Those portions of the metallic body that are to be boronized typically are covered with a layer of boronizing agent that is about 10 to about 20 mm in thickness. The amount of boronizing agent used in the process typically provides an amount of boron that exceeds the amount required to produce a boronized layer having the desired thickness.

Any suitable portion of the metallic body of the medical implant or medical implant part can be contacted with the boron produced by the boronizing agent. Preferably, the portion of the metallic body that is contacted with the boron produced by the boronizing agent corresponds to a bearing surface disposed on the outer surface of the body of the implant or implant part. When only limited portions of the metallic body of the implant or implant part are to be boronized, a masking agent can be applied to those portions of the metallic body that are not be boronized. Suitable masking agents include, but are not limited to, silicon carbide, asbestos, copper, aluminum oxide, tapes (e.g., Tesa tape No. 4541), or commercially available boronizing masking agents, such as EKrit. In certain embodiments, substantially all or all of the outer surface of the metallic body can be contacted with the boron produced by the boronizing agent.

The process of the invention comprises the step of heating the boronizing agent to a temperature at which the boronizing agent yields boron. The process of the invention also comprises the step of heating the medical implant or medical implant part to an elevated temperature for a time sufficient for at least a portion of the boron produced by the boronizing agent to diffuse into at least a portion of the metallic body of the medical implant or medical implant part. While the boronizing agent and the implant or implant part can be heated to different temperatures, the implant or the implant part and the boronizing agent typically are heated to substantially the same temperature. For example, when the boronizing agent is provided in the form of a liquid, paste, or solid, the boronizing agent typically is applied to the surface of the implant or implant part that is to be boronized, and the implant or implant part and the boronizing agent are together heated to a temperature at which the boronizing agent yields boron. Alternatively, the boronizing agent can be heated separately from the medical implant or medical implant part. In such a process, a carrier gas (e.g., an inert carrier gas such as nitrogen) typically is used to transport at least a portion of the boron produced by the boronizing agent to the surface of the metallic body so that the boron can diffuse into the metallic body. For example, U.S. Pat. No. 4,404,045 describes a process in which the boronizing agent is heated separately from the pieces to be boronized.

The medical implant or medical implant part and the boronizing agent can be heated to any suitable temperature. As will be understood by those of ordinary skill in the art, the temperatures suitable for the steps of the process will depend, at least in part, on the identity of the metal or metal alloy present in the metallic body and the particular boronizing agent used in the process. Preferably, the medical implant or medical implant part and the boronizing agent are heated to a temperature of about 500° C. or more (e.g., about 550° C. or more, about 580° C. or more, about 800° C. or more, or about 900° C. or more). Typically, the implant or implant part and the boronizing agent are heated to a temperature that is below the solidus of the metal or metal alloy (i.e., the temperature at which the metal or metal alloy begins to melt and comprises a mixture of solid and liquid phases). Preferably, the medical implant or medical implant part and the boronizing agent are heated to a temperature of about 1300° C. or less (e.g., about 1250° C. or less, about 1240° C. or less, or about 1100° C. or less). In certain embodiments, the medical implant or medical implant part and the boronizing agent preferably are heated to a temperature of about 850° C. to about 1100° C. (e.g., about 900° C. to about 1050° C., or about 1000° C.)

As will be understood by those of ordinary skill in the art, heating the medical implant or medical implant part to certain temperatures can negatively impact the stability of the metal or metal alloy from which the metallic body of the implant or implant part is comprised. For example, when the medical implant or medical implant part comprises Co28Cr6Mo alloy, heating the implant or implant part to a temperature of about 650° C. to about 1170° C. can lead to the formation of a multiple-phase solid region in the implant or implant part in which carbides can precipitate as the microstructure ages at the elevated temperature. While the precipitation of such carbides can increase the yield strength and hardness of the alloy, the precipitation also can decrease the ductility and toughness of the alloy, which effects can become significant when the implant or implant part is heated to a temperature of about 800° C. to about 1170° C. Such a change in the mechanical properties of the alloy may be acceptable for many applications (e.g., femoral heads), but may pose a concern where the implant or implant part has a relatively thin cross section and experiences loading that produces cyclic tensile stresses (e.g., the femoral component of a knee arthroplasty). Accordingly, in certain embodiments, such as when the metallic body of the medical implant or medical implant part comprises Co28Cr6Mo alloy and maintaining the ductility of the cobalt-chromium substrate is of particular concern, the medical implant or medical implant part and the boronizing agent preferably are heated to a temperature of about 550° C. to about 800° C., more preferably about 650° C. to about 800° C. Alternatively, the medical implant or medical implant part and the boronizing agent can be heated to a temperature of about 1180° C. to about 1250° C., preferably about 1200° C. to about 1240° C. However, when the implant or implant part comprises a cobalt-chromium alloy (e.g., Co28Cr6Mo alloy) and it is heated to a temperature of about 1180° C. to about 1250° C., the implant or implant part preferably is rapidly cooled to a temperature of about 800° C. or less after the desired amount of boron has diffused into the metallic body of the medical implant or medical implant part. While not wishing to be bound to any particular theory, it is believed that rapidly cooling the implant or implant part to a temperature of about 800° C. or less will avoid significant carbide precipitation in the implant or implant part.

In another embodiment, the medical implant or medical implant part and the boronizing agent can be heated to a temperature of about 800° C. to about 1170° C. While such a temperature range may lead to the precipitation of carbides in an implant or implant part comprising a cobalt-chromium alloy (e.g., Co28Cr6Mo alloy), the medical implant or medical implant part, if desired, can be subjected to further processing to ensure that the ductility and toughness of the implant or implant part are not significantly negatively affected. In one such embodiment, such as when the metallic body of the medical implant or medical implant part comprises a cobalt-chromium alloy (e.g., Co28Cr6Mo) and maintaining the ductility of the cobalt-chromium substrate is of particular concern, the process preferably further comprises the steps of (f) heating the medical implant or medical implant part to a temperature of about 1200° C. to about 1240° C. after at least a portion of the boron produced by the boronizing agent has diffused into the metallic body of the medical implant or medical implant part, and (g) rapidly cooling the medical implant or medical implant part to a temperature of about 800° C. or less. While not wishing to be bound to any particular theory, it is believed that heating the medical implant or medical implant part to a temperature of about 1200° C. to about 1240° C. will dissolve any carbides that may have precipitated while the implant or implant part was heated to a temperature of about 800° C. to about 1170° C. Furthermore, it is believed that rapidly cooling the implant or implant part to a temperature of about 800° C. or less will avoid any further significant carbide precipitation in the implant or implant part.

The medical implant or medical implant part and the boronizing agent can be heated in any suitable environment. In order to reduce the potential oxidation of the metal or metal alloy contained in the implant or implant part, the implant or implant part and the boronizing agent preferably are heated in a vacuum or reduced pressure atmosphere, an inert atmosphere, or a reducing atmosphere. For example, the implant or implant part and the boronizing agent can be heated in an inert gaseous atmosphere, such as an atmosphere comprising argon, nitrogen, or any suitable combination of inert gases. Alternatively, the implant or implant part and the boronizing agent can be heated in a reducing gaseous atmosphere, such as an atmosphere comprising hydrogen, dissociated ammonia, forming gas (e.g., a gas containing about 5-30% hydrogen and about 70-95% nitrogen), hydrocarbons, a mixtures of at least two of the aforementioned reducing gases, or a mixture of at least one reducing gas with at least one inert gas.

The medical implant or medical implant part is maintained at an elevated temperature for a time sufficient for at least a portion of the boron produced by the boronizing agent to diffuse into at least a portion of the metallic body of the implant or implant part. As will be understood by those of ordinary skill in the art, the amount of time necessary for the boron to diffuse into the metallic body of the implant or implant part will depend upon several factors, such as the type of metal or metal alloy present in the body, the temperature(s) to which the boronizing agent and the implant or implant part are heated, and the desired thickness of the resulting boronized layer. Typically, the medical implant or implant part is maintained at the elevated temperature (e.g., the temperature to which the implant or implant part and the boronizing temperature were heated so that the boronizing agent would yield boron to diffuse into the implant or implant part) for about 30 minutes or more, preferably about 60 minutes or more, and more preferably about 120 minutes or more. Typically, the medical implant or implant part is maintained at the elevated temperature for about 720 minutes or less (e.g., about 660 minutes or less), preferably about 600 minutes or less, and more preferably about 540 minutes or less (e.g., about 500 minutes or less, or about 480 minutes or less). However, those of ordinary skill in the art will readily understand that the amount of time necessary to produce a boronized layer having a desired thickness may be longer when relatively low temperatures are used (e.g., about 850° C. or less) or a relatively thick (e.g., about 8 μm or more) boronized layer is desired.

The characteristics of the medical implant or medical implant part produced by the process of the invention (e.g., the composition of the metallic body, the composition of the boronized layer, the thickness of the boronized layer, the hardness of the boronized layer, etc.) can be the same as those set forth above for the medical implant or medical implant part of the invention.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A medical implant or medical implant part comprising:
(a) a metallic body comprising a metal or metal alloy, and
(b) a bearing surface disposed on the body, the bearing surface comprising a boronized layer of the metal or metal alloy.

2. The medical implant or medical implant part of claim 1, wherein the metallic body comprises a metal or metal alloy selected from the group consisting of cobalt, cobalt alloys, titanium, titanium alloys, and mixtures thereof.

3. The medical implant or medical implant part of claim 2, wherein the metal or metal alloy is selected from the group consisting of cobalt, cobalt-chromium alloys, titanium, titanium-aluminum alloys, and mixtures thereof.

4. The medical implant or medical implant part of claim 3, wherein the cobalt-chromium alloy is Co28Cr6Mo.

5. The medical implant or medical implant part of claim 3, wherein the titanium-aluminum alloy is selected from the group consisting of Ti3Al2.5V and Ti6Al4V.

6. The medical implant or medical implant part of claim 1, wherein the boronized layer comprises borides having the formula MeB, MeB₂, or Me₂B, wherein Me represents a metal present in the body of the medical implant or medical implant part.

7. A medical implant for implantation into a patient, the medical implant comprising:
(a) a femoral component for replacing one or more of the patient's femoral condyles, the femoral component having a metallic body comprising a metal or metal alloy and a bearing surface disposed on the body, the bearing surface comprising a boronized layer of the metal or metal alloy,
(b) a tibial component for replacing at least a portion of the patient's proximal tibial articular surface, and
(c) a polymeric bearing component which rests on the tibial component and confronts the bearing surface of the femoral component.

8. The medical implant of claim 7, wherein the metallic body of the femoral component comprises a metal or metal alloy selected from the group consisting of cobalt, cobalt alloys, titanium, titanium alloys, and mixtures thereof.

9. The medical implant of claim 8, wherein the metal or metal alloy is selected from the group consisting of cobalt, cobalt-chromium alloys, titanium, titanium-aluminum alloys, and mixtures thereof.

10. The medical implant of claim 9, wherein the cobalt-chromium alloy is Co28Cr6Mo.

11. The medical implant of claim 9, wherein the titanium-aluminum alloy selected from the group consisting of Ti3Al2.5V and Ti6Al4V.

12. The medical implant of claim 7, wherein the boronized layer comprises borides having the formula MeB, MeB₂, or Me₂B, wherein Me represents a metal present in the body of the medical implant or medical implant part.

13. A medical implant for implantation into a patient, the medical implant comprising:
(a) a femoral stem for anchoring the implant into the patient's femur,
(b) a femoral head which attaches to the upper end of the femoral stem, the femoral head having a metallic body comprising a metal or metal alloy and a bearing surface disposed on the body, the bearing surface comprising a boronized layer of the metal or metal alloy, and
(c) an acetabular component for replacing the patient's acetabulum, the acetabular component comprising a liner which confronts the bearing surface of the femoral head.

14. The medical implant of claim 13, wherein the metallic body of the femoral head comprises a metal or metal alloy selected from the group consisting of cobalt, cobalt alloys, titanium, titanium alloys, and mixtures thereof.

15. The medical implant of claim 14, wherein the metal or metal alloy is selected from the group consisting of cobalt, cobalt-chromium alloys, titanium, titanium-aluminum alloys, and mixtures thereof.

16. The medical implant of claim 15, wherein the cobalt-chromium alloy is Co28Cr6Mo.

17. The medical implant of claim 15, wherein the titanium-aluminum alloy selected from the group consisting of Ti3Al2.5V and Ti6Al4V.

18. The medical implant of claim 13, wherein the boronized layer comprises borides having the formula MeB, MeB₂, or Me₂B, wherein Me represents a metal present in the body of the medical implant or medical implant part.

19. The medical implant of claim 13, wherein the liner is comprised of a metal or metal alloy.

20. The medical implant of claim 19, wherein the portion of the liner which confronts the bearing surface of the femoral head comprises a boronized layer of the metal or metal alloy of which the liner is comprised.

21. A process for producing a medical implant or medical implant part, the process comprising the steps of:
(a) providing a medical implant or medical implant part having a metallic body,
(b) providing a boronizing agent which yields boron upon heating,
(c) heating the boronizing agent to a temperature at which the boronizing agent yields boron,
(d) contacting at least a portion of the metallic body with the boron produced by the boronizing agent, and
(e) heating the medical implant or medical implant part to an elevated temperature for a time sufficient for at least a portion of the boron produced by the boronizing agent to diffuse into at least a portion of the metallic body of the medical implant or medical implant part.

22. The process of claim 21, wherein the metallic body comprises a metal or metal alloy selected from the group consisting of cobalt, cobalt alloys, titanium, titanium alloys, and mixtures thereof.

23. The process of claim 22, wherein the metal or metal alloy is selected from the group consisting of cobalt, cobalt-chromium alloys, titanium, titanium-aluminum alloys, and mixtures thereof.

24. The process of claim 23, wherein the cobalt-chromium alloy is Co28Cr6Mo.

25. The process of claim 23, wherein the titanium-aluminum alloy is selected from the group consisting of Ti3Al2.5V and Ti6Al4V.

26. The process of claim 21, wherein the medical implant or medical implant part and the boronizing agent are heated to a temperature of about 550° C. to about 1300° C.

27. The process of claim 24, wherein the medical implant or medical implant part and the boronizing agent are heated to a temperature of about 550° C. to about 800° C.

28. The process of claim 27, wherein the medical implant or medical implant part and the boronizing agent are heated to a temperature of about 650° C. to about 800° C.

29. The process of claim 24, wherein the medical implant or medical implant part and the boronizing agent are heated to a temperature of about 800° C. to about 1170° C.

30. The process of claim 29, wherein the process further comprises the steps of:
(f) heating the medical implant or medical implant part to a temperature of about 1200° C. to about 1240° C. after at least a portion of the boron produced by the boronizing agent has diffused into the metallic body of the medical implant or medical implant part, and
(g) rapidly cooling the medical implant or medical implant part to a temperature of about 800° C. or less.

31. The process of claim 24, wherein the medical implant or medical implant part and the boronizing agent are heated to a temperature of about 1180° C. to about 1250° C.

32. The process of claim 31, wherein the medical implant or medical implant part and the boronizing agent are heated to a temperature of about 1200° C. to about 1240° C.
